(19) United States
(12) Patent Application Publication
(10) Pub. No.: US 2010/0125223 A1
(43) Pub. Date: May 20, 2010

(54) CELL SAMPLING DEVICE

(76) Inventor: Cheng-Hsiung Roan, Taipei (TW)

Correspondence Address: PAI PATENT & TRADEMARK LAW FIRM, 1001 FOURTH AVENUE, SUITE 3200, SEATTLE, WA, 98154, US

(21) Appl. No.: 12/270,870

(22) Filed: Nov. 14, 2008

(51) Int. Cl. A61B 10/00

(52) U.S. Cl. 600/569

(57) ABSTRACT

A cell sampling device is adapted for collecting the cells to be examined from the oral cavity or other organs for medical use. The device includes a handle and a brush having clusters of bristles in a rectangular shape connected to the end of the handle. In particular, the handle has a breaking portion formed at one side of the handle, two opposite sides of the handle or around the handle near the brush. The handle is turned to collect the cells to be examined. The breaking portion of the handle can be broken to dispose the unnecessary section of the handle. Thus, the device is able to be stored in a container for convenient storage of the cells to be examined.

### BACKGROUND OF THE INVENTION

(a) Field of the Invention

The present invention relates to a cell sampling device, and more particular to a medical sampling device having a brush to collect the cells to be examined for a subsequent examination.

(b) Description of the Prior Art

It is improper for a doctor in a clinic or hospital to directly collect the cells to be examined of a patient and to make an examination, test or analysis. The doctor uses a sampling device to collect the cells to be examined at a specific organ of the patient, and send the cells to a laboratory or examination center.

A conventional sampling device has a handle, a brush head at one end of the handle, and a line of bristles at the other end of the brush head. The doctor can hold the handle and extend it to the oral cavity, vagina or other organs for sampling. The soft brush is moved back and forth at a chosen location to collect the cells to be examined. The cells to be examined can be transferred to a laboratory or an examination center for making an examination or analysis.

The handle of the conventional sampling device has a specific length for the brush extending into the oral cavity or vagina. After collecting the cells to be examined, the long handle becomes a burdensome portion, influencing the seal, transfer and storage of the cells to be examined. Besides, the brush of the convention sampling device is arranged in a single line. The operating way is to move back and forth for collecting the cells to be examined. This is not easy to operate in the deep of the oral cavity or vagina.

### SUMMARY OF THE INVENTION

The primary object of the present invention is to provide a cell sampling device so that a doctor can hold and rotate a handle for collecting the cells. Compared to the back and forth movement of the prior art, the present invention is convenient to collect the cells to be examined. After collecting the cells, the unnecessary section of the handle can be broken in a rapid way for putting the device into a container. This provides a convenient way in use.

According to the aforesaid object of the present invention, there is provided a cell sampling device comprising a handle, a brush head, and a brush. The handle is a rectangular or circular rod coupled to a first end of the brush head. The brush is connected to a send second of the brush head. The brush has clusters of bristles in a rectangular shape connected to the end of the handle. The handle has a breaking portion formed at one side of the handle, two opposite sides of the handle or around the handle near the brush. Thus, a doctor can hold and rotate a handle for collecting the cells to be examined. The breaking portion of the handle can be broken to dispose the unnecessary section of the handle. The brush head and the brush are stored in a container for convenient storage of the cells to be examined.

### BRIEF DESCRIPTION OF THE DRAWINGS

FIG. 1 is a perspective view of a cell sampling device having a rectangular handle of the present invention;

FIG. 2 is a perspective view showing a breaking portion of the rectangular handle according to a preferred embodiment of the present invention;

FIG. 3 is a perspective view showing the breaking portion of the rectangular handle according to another preferred embodiment of the present invention;

FIG. 4 is a perspective view of a cell sampling device having a circular handle of the present invention;

FIG. 5 is a perspective view showing a breaking portion of the circular handle according to a preferred embodiment of the present invention;

FIG. 6 is a perspective view showing the breaking portion of the circular handle according to another preferred embodiment of the present invention;

FIG. 7 is a perspective view of the present invention in an operating status;

FIG. 8 is bottom view of the present invention in an operating status;

FIG. 9 is a perspective view showing that the breaking portion of the present invention is broken;

FIG. 10 is a schematic view of the present invention showing a brush head and a brush in an operating status; and

FIG. 11 is a schematic view of the present invention showing the brush head and the brush in a stored status.

### DETAILED DESCRIPTION OF THE PREFERRED EMBODIMENTS

Embodiments of the present invention will now be described, by way of example only, with reference to the accompanying drawings. The present invention relates to a cell sampling device for medical use, which comprises a handle 1, a brush head 2, and a brush 3.

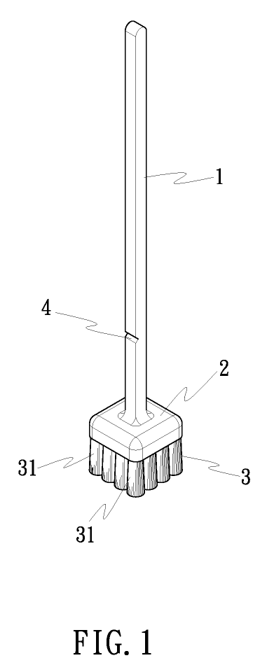

As shown in FIG. 1, the handle 1 is a rectangle rod as a holding portion when sampling cells. One end of the handle 1 is coupled to a first end of the brush head 2. The handle 1 and the brush head 2 may be molded as one piece. The brush head 2 is a block in a rectangular or other shape for connection of the brush 3. The brush head 2 is coupled to the handle 1. The brush 3 is made of soft material and connected to a second end of the brush head 2.

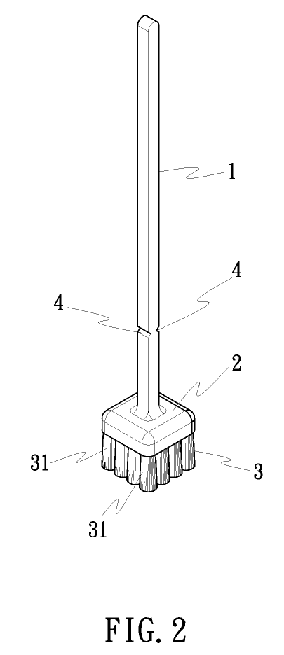

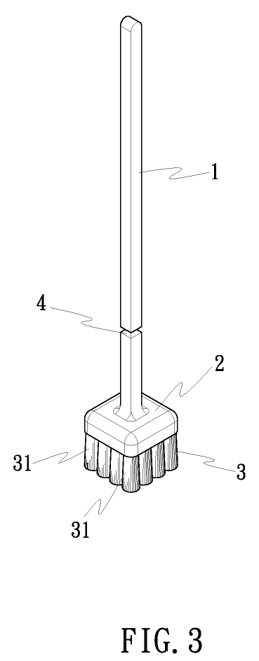

The improvement of the present invention is that the brush 3 comprises clutches of bristles 31 arranged in a rectangular shape coupled to the end of the brush head 2. As shown in the drawings, the bristles 31 may be arranged in four lines and four rows or in three lines and three rows. The handle 1 has a breaking portion 4 near the first end of the brush head 2. The breaking portion 4 may be a V-shaped notch at one side of the handle 1, two notches at two opposite sides of the handle 1 as shown in FIG. 2, or a circular notch around the handle 1 as shown in FIG. 3.

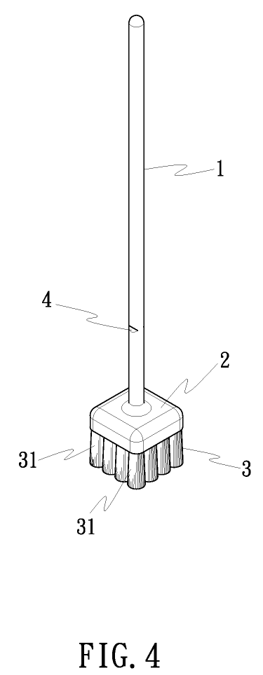

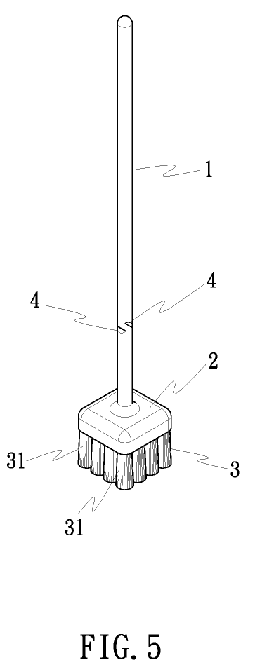

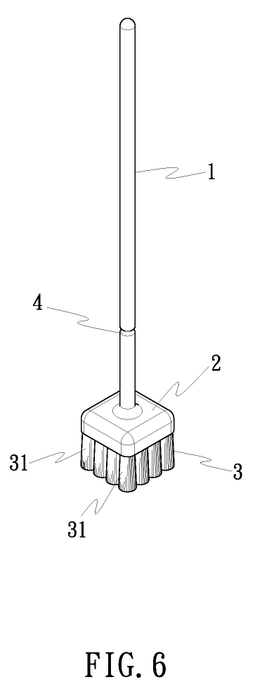

As shown in FIG. 4, the handle 1 of the cell sampling device of the present invention may be a circular rod. The breaking portion 4 may be a V-shaped notch at one side of the handle 1 as shown in FIG. 4, two notches at two opposite sides of the handle 1 as shown in FIG. 5, or a circular notch around the handle 1 as shown in FIG. 6. The brush 3 and other construction are the same as the aforesaid.

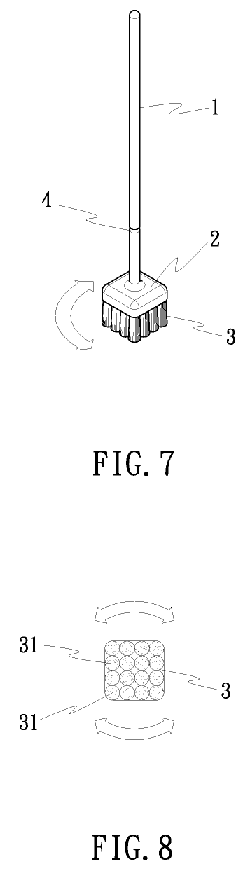

By the improved design of the aforesaid cell sampling device, a doctor can hold the handle 1 and extend the device into the oral cavity or the vagina of a patient for collecting the cells to be examined. The handle 1 is turned for the end of the brush 3 to collect the cells to be examined as shown in FIGS. 7 and 8. Compared to the back and forth movement of the prior art, the present invention is convenient to collect the cells to be examined.

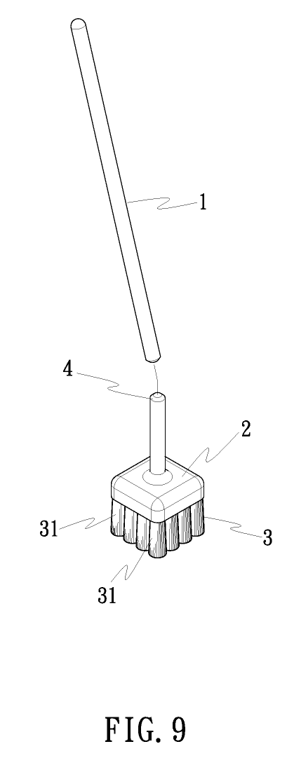

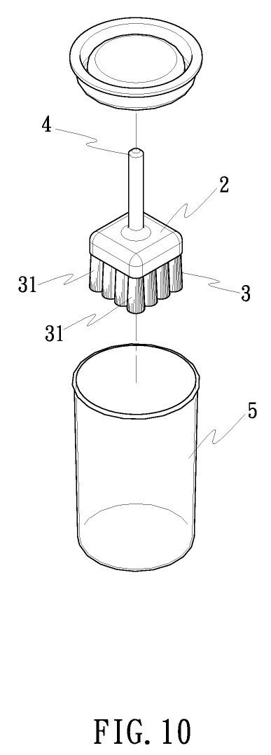

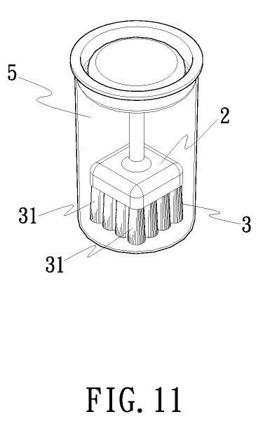

When the brush 3 gets the cells to be examined, the breaking portion 4 will be broken away in a rapid way to save strength. The unnecessary section of the handle 1 is disposable as shown in FIG. 9. The brush head 2 and the brush 3 are placed into a container 5 and then sealed, as shown in FIGS. 10 and 11. The cells to be examined can be transferred to a laboratory or an examination center for making an examination or analysis. Accordingly, the improved design of the present invention has a smaller size and is easy to operate and break the handle, providing a simple and convenient way in use.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A cell sampling device, comprising a handle coupled to a first end of a brush head and a brush connected to a second end of the brush head, characterized by: the brush comprising clusters of bristles in a rectangular shape connected to the brush head, the handle has a breaking portion near the first end of the brush head.

2. The cell sampling device as claimed in claim 1, wherein the breaking portion is formed at one side of the handle near the first end of the brush head.

3. The cell sampling device as claimed in claim 1, wherein the breaking portion is formed at two opposite sides of the handle near the first end of the brush head.

4. The cell sampling device as claimed in claim 1, wherein the breaking portion is a circular notch around the handle near the first end of the brush head.

5. The cell sampling device as claimed in claim 1, wherein the breaking portion is a V-shaped notch.

6. The cell sampling device as claimed in claim 1, wherein the clusters of bristles are arranged in four lines and four rows.

7. The cell sampling device as claimed in claim 1, wherein the clusters of bristles are arranged in three lines and three rows.
